(19) United States
(12) Patent Application Publication
(10) Pub. No.: US 2026/0306311 A1
(43) Pub. Date: Oct. 8, 2026

(54) ABSORBENT ARTICLE

(71) Applicant: LIVEDO CORPORATION, Ehime (JP)

(72) Inventors: Yoshiko IWAMOTO, Tokushima (JP); Yuki TAKAHASHI, Tokushima (JP)

(73) Assignee: LIVEDO CORPORATION, Ehime (JP)

(21) Appl. No.: 19/453,702

(22) Filed: Jan. 20, 2026

(30) Foreign Application Priority Data
Jan. 31, 2025 (JP) 2025-015570

(51) Int. Cl. A61F 13/511; A61F 13/58

(52) U.S. Cl. CPC A61F 13/51108; A61F 13/51121; A61F 13/58

(57) ABSTRACT

An absorbent article (1) including a top sheet (2), a back sheet (3) and an absorbent body (4) disposed therebetween. The top sheet (2) has thick parts (21) and thin parts (22) extending in parallel in the front-rear direction and alternately arranged in the width direction. The thin parts (22) are recessed relative to the thick parts (21) on both skin-facing and non-skin-facing surfaces (23, 24) of the top sheet (2). The thick and thin parts (21, 22) extend from a portion overlapping with the absorbent body (4) and extend forward beyond a front edge and extend rearward beyond a rear edge of the absorbent body (4). The non-skin-facing surface (24) has a first adhesive region (25) in that adhesive is spirally or meanderingly arranged and a second adhesive region (26) over that adhesive is entirely arranged. The second adhesive region (26) is positioned anterior and posterior to the first adhesive region (25) and arranged at least forward and rearward of the absorbent body (4).

This application is related to and claims priority under 35 U.S.C. 119 to Japanese patent application No. 2025-015570, filed on Jan. 31, 2025.

### TECHNICAL FIELD

The present invention relates to an absorbent article such as urine pads (including light incontinence pads), sanitary napkins and disposable diapers.

### BACKGROUND OF THE INVENTION

Various top sheets used in absorbent articles are known. For example, Patent Document 1 discloses a nonwoven fabric having ridges and grooves extending in parallel in the front-rear direction, and being alternately arranged in the width direction, and Patent Document 2 discloses an absorbent article including a nonwoven fabric as a top sheet having ridges and grooves alternately arranged in the width direction.

### CITATION LIST

### Patent document

### Patent Document 1

Japanese Unexamined Laid-open Patent Application Publication No. 2010-285735

### Patent Document 2

Japanese Unexamined Laid-open Patent Application Publication No. 2017-038838

### SUMMARY OF INVENTION

### Technical Problem

An absorbent article is handled by grasping the front and rear end portions of the absorbent article when attaching it to underwear or placing it on a wearer's crotch, and at this time, the absorbent article is expected to retain its shape and stay as flat as possible, which improves the handleability of the absorbent article. Furthermore, after the absorbent article is placed on the wearer's crotch, it is expected to have sufficient absorbency of bodily fluids such as urine. The present invention has been made in view of the above circumstances, and an object of the present invention is to provide an absorbent article that is easy to handle and has sufficient absorbency for bodily fluids such as urine.

### Solution to Problem

The absorbent article of the present invention, which achieves the above object, is as follows.
[1] An absorbent article having a front-rear direction and a width direction, including a top sheet, a back sheet and an absorbent body disposed between the top sheet and the back sheet. The top sheet includes thick parts and thin parts that extend in parallel in the front-rear direction, and are arranged alternately in the width direction. The thin parts are recessed relative to the thick parts on a skin-facing surface of the top sheet, and the thin parts are recessed relative to the thick parts on a non-skin-facing surface of the top sheet. The thick parts and the thin parts extend from a portion overlapping with the absorbent body and extend forward beyond a front edge of the absorbent body and extend rearward beyond a rear edge of the absorbent body. The non-skin-facing surface of the top sheet includes a first adhesive region in which adhesive is arranged to extend in a spiral pattern or a meandering pattern in the front-rear direction, and a second adhesive region over which adhesive is entirely arranged. The first adhesive region is arranged at least to overlap with the absorbent body, and the second adhesive region is positioned anterior and posterior to the first adhesive region, and is arranged at least forward of the front edge of the absorbent body and at least rearward of the rear edge of the absorbent body.
[2] The absorbent article according to [1] above, wherein the second adhesive region is arranged from a portion that overlaps with at least one of a front end part and a rear end part of the absorbent body.
[3] The absorbent article of [2] above, in the second adhesive region that is arranged from the portion overlapping with the at least one of the front end part and the rear end part of the absorbent body, wherein the length in the front-rear direction of the overlapping portion is 20% or shorter of the length in the front-rear direction of the absorbent body.
[4] The absorbent article according to any one of [1] to [3] above, wherein the second adhesive region includes a first region extending at least from the front edge of the absorbent body to a front edge of the top sheet and a second region extending at least from the rear edge of the absorbent body to a rear edge of the top sheet.
[5] The absorbent article according to any one of [1] to [4] above, wherein the front edge of the absorbent body is straight and the rear edge of the absorbent body is straight.
[6] The absorbent article according to any one of [1] to [5] above, wherein the absorbent body includes two or more sheet members, and an absorbent polymer without a pulp fiber is disposed between the sheet members, the absorbent body includes a sealed part in which the sheet members are bonded to each other and a non-sealed part in which the sheet members are not bonded to each other, and the absorbent polymer is disposed in the non-sealed part, and the sealed part and the non-sealed part extend in parallel in the front-rear direction and are arranged alternately in the width direction.
[7] The absorbent article according to any one of [1] to [6] above, wherein the top sheet is a nonwoven fabric, and the nonwoven fabric is heat-embossed at the thin parts.
[8] The absorbent article according to any one of [1] to [7] above, wherein the thick parts and the thin parts extend continuously in the front-rear direction from a front edge to a rear edge of the top sheet.

### Advantageous Effects of Invention

In the absorbent article according to an embodiment of the present invention, the top sheet includes thick parts and thin parts extending from a portion that overlaps with the absorbent body and extending forward beyond a front edge and extending rearward beyond a rear edge of the absorbent body, and includes a second adhesive region, over which adhesive is entirely arranged, provided at least forward of the front edge of the absorbent body and rearward of the rear edge of the absorbent body on a non-skin-facing surface of the top sheet. Therefore, shape retention of the absorbent article in a planar direction at the front end portion and the rear end portion is enhanced. Accordingly, it is easier for a user to grasp the front and rear end portions of the absorbent article 1, and the absorbent article 1 tends to be maintained in a flat state while it is grasped at the front and rear end portions, resulting in better handleability of the absorbent article. Furthermore, in the absorbent article according to an embodiment of the present invention, the surface pattern of the thick parts and the thin parts of the top sheet appear to be more pronounced in the second adhesive region over which the adhesive is entirely arranged, and therefore, it is easy to visually distinguish between the portion where the absorbent body is present and the region where the absorbent body is not present when handling the absorbent article, making it easier to accurately position the absorbent article on underwear or a crotch. In contrast, since in the first adhesive region on the non-skin-facing surface of the top sheet, adhesive is arranged to extend in a spiral pattern or a meandering pattern in the front-rear direction, the top sheet tends to be more likely to develop wrinkles extending in the width direction in the first adhesive region. As a result, in the first adhesive region, both the diffusion of urine and the like in front-rear direction and the diffusion in the width direction are ensured on the skin-facing surface of the top sheet. Since the first adhesive region is provided so as to overlap with the absorbent body, urine and the like excreted on the skin-facing surface of the top sheet are more likely to diffuse in both the front-rear direction and the width direction in the overlapping portion with the absorbent body, allowing the urine and the like to rapidly pass through the top sheet and be suitably absorbed by the absorbent body.

### BRIEF DESCRIPTION OF THE DRAWINGS

FIG. 1 illustrates a plan view of a light incontinence pad, which is an example of an absorbent article of the present invention, as seen from a top sheet side thereof.

FIG. 2 illustrates a widthwise cross-sectional view of a top sheet included in the absorbent article shown in FIG. 1.

FIG. 3 illustrates a cross-sectional view taken along a line III-III of the absorbent article shown in FIG. 1.

FIG. 4 illustrates a schematic plan view of an adhesive coating arrangement with adhesive regions on a non-skin-facing surface of the top sheet in the absorbent article shown in FIG. 1.

FIG. 5 illustrates an example of an absorbent body included in the absorbent article shown in FIG. 1, and shows a widthwise cross-sectional view of the absorbent body.

FIG. 6 illustrates a partial cutaway plan view of the absorbent body shown in FIG. 5 as seen from a top sheet side thereof.

### DETAILED DESCRIPTION OF EMBODIMENTS

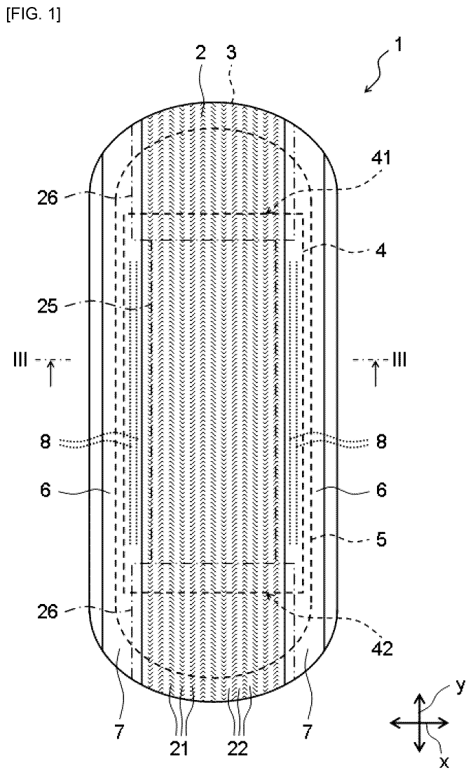

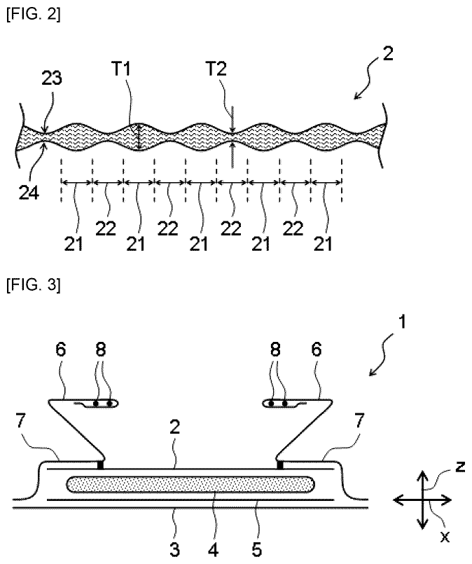

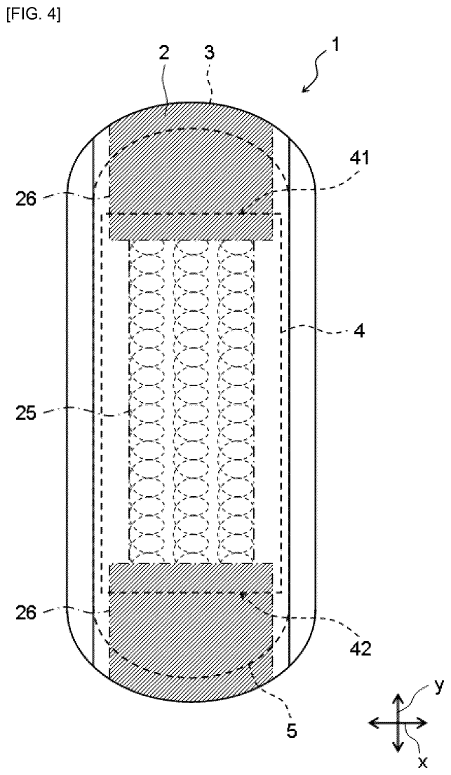

The example of an absorbent article of the present invention is explained with reference to drawings. However, the present invention is not limited to embodiments shown in the drawings. FIGS. 1 to 4 show a configuration example of a light incontinence pad as an embodiment of an absorbent article of the present invention. As used herein, the singular forms “a,” “an,” and “the” include plural referents unless the context clearly dictates otherwise. FIG. 1 shows a plan view of the absorbent article (a light incontinence pad) as seen from a top sheet side thereof. FIG. 2 shows a widthwise cross-sectional view of a top sheet included in the absorbent article shown in FIG. 1. FIG. 3 shows a cross-sectional view taken along a line III-III of the absorbent article shown in FIG. 1. FIG. 4 shows a schematic plan view of an adhesive coating arrangement with adhesive regions on a non-skin-facing surface of the top sheet after side sheets are removed from the absorbent article shown in FIG. 1. Absorbent articles according to the present invention are not limited to embodiments shown in the drawings.

An absorbent article 1 includes a top sheet 2, a back sheet 3 and an absorbent body 4 disposed between the top sheet 2 and the back sheet 3. The top sheet 2 is disposed at a side closer to a skin-facing surface of the absorbent body 4, and the back sheet 3 is disposed at a side closer to a non-skin-facing surface of the absorbent body 4. Excrement that has passed through the top sheet 2 would be absorbed into the absorbent body 4. The back sheet 3 prevents excrement from leaking to the outside. Embodiments of the absorbent article 1 can be applied to, for example, urine pads (including light incontinence pads), sanitary napkins, disposable diapers, and the like, and are particularly suitable for light incontinence pads or sanitary napkins.

The absorbent article 1 has a front-rear direction y and a width direction x. The front-rear direction y indicates a direction extending in a front-rear direction at a crotch of a wearer when the absorbent article is worn. The width direction x indicates a direction orthogonal to the front-rear direction y on the same plane as the absorbent article 1. The width direction x corresponds to a left-right direction of a wearer when the absorbent article is worn. Furthermore, a direction parallel to the plane defined by the front-rear direction y and the width direction x is referred to as a planar direction, and a direction orthogonal to this plane is defined as a thickness direction z. The absorbent article 1 has a skin-facing surface and a non-skin-facing surface that are defined in the thickness direction z.

The top sheet 2 is provided so as to face a wearer's skin when the absorbent article 1 is worn. The top sheet 2 is preferably liquid permeable. As the top sheet 2, a nonwoven fabric made from hydrophilic fibers such as cellulose, rayon and cotton; a nonwoven fabric which is formed from hydrophobic fibers such as polyolefin (e.g., polypropylene or polyethylene), polyester (e.g., PET) and polyamide (e.g., nylon), and in which the hydrophobic fibers are hydrophilized with a surfactant on the surface thereof; or the like can be used, for example.

The back sheet 3 is preferably liquid impermeable. As the back sheet 3, a nonwoven fabric made from hydrophobic fibers such as polyolefin (e.g., polypropylene or polyethylene), polyester (e.g., PET) and polyamide (e.g., nylon); a plastic film; or the like can be used, for example. Also, as the back sheet 3, a laminate of a nonwoven fabric and a plastic film may be used.

The top sheet 2 and back sheet 3 are preferably longer than the absorbent body 4 in the front-rear direction y. Specifically, the top sheet 2 and the back sheet 3 are preferably arranged to extend forward beyond a front edge 41 and extend rearward beyond a rear edge 42 of the absorbent body 4.

The absorbent article 1 may include a base sheet 5 provided between the absorbent body 4 and the back sheet 3. Providing the base sheet 5 may improve shape retention of the absorbent article 1.

The absorbent article 1 preferably includes standing flaps 6 provided on both sides in the width direction x of the skin-facing surface of the top sheet 2. By providing the standing flap 6, lateral leakage of urine and the like is prevented. The standing flaps can be formed, for example, by adhering side sheets 7 that extend in the front-rear direction y to both sides in the width direction x of the top sheet 2, and providing elastic members 8 for the standing flaps on an inner portion of the side sheets 7 in the width direction x. By providing the side sheets 7 and the elastic members 8 for the standing flaps in this manner, the inner portions of the side sheets 7 in the width direction x stand up toward a wearer's skin due to contraction force of the elastic members 8, thereby forming the standing flaps 6. The standing flaps 6 or the side sheets 7 may be made of a material preferably selected from liquid-impermeable plastic films, liquid-impermeable nonwoven fabrics, and a combination selected among the liquid-impermeable plastic films and the liquid-impermeable nonwoven fabrics.

As nonwoven fabrics used for each sheet described above, a spunbond nonwoven fabric, a pointbond nonwoven fabric, an air-through nonwoven fabric, a meltblown nonwoven fabric, an airlaid nonwoven fabric, a spunlace nonwoven fabric, an SMS nonwoven fabric, or the like can be used. The mass per unit area of each of these sheets is preferably 8 g/m² or more, more preferably 10 g/m² or more, and preferably 40 g/m² or less.

The absorbent body 4 is not particularly limited as long as it contains an absorbent material that is able to absorb bodily exudates such as urine. As the absorbent body 4, a shaped product of an absorbent material, which is formed into a certain shape, may be used, or the shaped product wrapped with a cover sheet selectable among paper (e.g., tissue paper and thin paper) and liquid-permeable nonwoven fabrics may be used. Examples of the absorbent material include, for example, a hydrophilic fiber such as a cellulose fiber, and an absorbent polymer such as a polyacrylic absorbent polymer, a polyasparaginic absorbent polymer, a cellulosic absorbent polymer and a starch-acrylonitrile absorbent polymer. The absorbent material may include a thermally fusible fiber such as a polyolefin (e.g., polyethylene and polypropylene) fiber, a polyester (e.g., PET) fiber and a polyamide fiber. These thermally fusible fibers may be hydrophilized with a surfactant or the like to increase affinity with urine and the like.

The absorbent material preferably includes a hydrophilic fiber in view of increasing absorption rate of urine and the like. In addition, in view of enhancing absorption capacity, the absorbent material preferably includes an absorbent polymer. Therefore, as one example of the absorbent body 4, it may contain a hydrophilic fiber (especially a pulp fiber) and an absorbent polymer. In this case, the absorbent material to be used is preferably formed by mixing absorbent polymer particles with a hydrophilic-fiber assembly, or dispersing absorbent polymer particles on a hydrophilic-fiber assembly, for example. The absorbent body 4 may include two or more absorbent layers.

The absorbent body 4 may be a sheet-shaped absorbent body. Examples of the sheet-shaped absorbent body include one that is configured to contain an absorbent polymer but not contain a pulp fiber between sheet members. Such a sheet-shaped absorbent body can achieve a high absorption capacity since it contains an absorbent polymer between sheet members. Furthermore, since the sheet-shaped absorbent body does not contain a pulp fiber between sheet members, it can be formed thin and with reduced bulk.

The absorbent body 4 may include two or more sheet-shaped absorbent layers. For example, the absorbent body 4 may include a first sheet-shaped absorbent layer on a side closer to the top sheet 2 and a second sheet-shaped absorbent layer on a side closer to the back sheet 3. The number of absorbent layers included in the absorbent body 4 may be appropriately determined according to the application, and for example, it may have three or more sheet-shaped absorbent layers. Such an absorbent body 4 can provide an absorbent article with a higher absorption capacity while maintaining a thin profile, since the absorbent polymer is arranged in layers in the thickness direction as well as the planar direction.

A shape (planar shape) of the absorbent body 4 is not particularly limited. The shape of the absorbent body 4 may be determined as appropriate according to application, and examples of that include a substantially rectangular shape, an hourglass shape, a center nipped-in gourd shape, a battledore shape and others. In the case where the absorbent body 4 includes two or more absorbent layers, the shapes (planar shapes) and/or sizes of the absorbent layers may differ from each other.

The top sheet 2 includes thick parts 21 and the thin parts 22 that extend in parallel in the front-rear direction y and are alternately arranged in the width direction x. As shown in FIG. 2, the thin parts 22 are recessed relative to the thick parts 21 on a skin-facing surface 23 of the top sheet 2, and the thin parts 22 are recessed relative to the thick parts 21 on a non-skin-facing surface 24 of the top sheet 2.

The thick parts 21 and the thin parts 22 of the top sheet 2 extend from a portion overlapping with the absorbent body 4 and extend forward beyond a front edge 41 of the absorbent body 4 and extend rearward beyond a rear edge 42 of the absorbent body 4. The non-skin-facing surface 24 of the top sheet 2 includes a first adhesive region 25 in which adhesive is arranged to extend in a spiral pattern or a meandering pattern in the front-rear direction y, and a second adhesive region 26 over which adhesive is entirely arranged. The first adhesive region 25 is arranged at least to overlap with the absorbent body 4. The second adhesive region 26 is positioned anterior and posterior to the first adhesive region 25, and is arranged at least forward of the front edge of the absorbent body 4 and rearward of the rear edge of the absorbent body 4. The portion where the top sheet 2 overlaps with the absorbent body 4 and the regions, in that the top sheet 2 does not overlap with the absorbent body 4, and that are arranged at least forward of the front edge of the absorbent body 4 and rearward of the rear edge of the absorbent body 4 are able to be determined in a plan view seen from a side expected to face a wearer's skin, of the absorbent article 1.

In the top sheet 2, the thick parts 21 are formed to be thicker than the thin parts 22. The thick parts 21 are formed to protrude more than the thin parts 22 on both the skin-facing surface 23 and the non-skin-facing surface 24 of the top sheet 2; that is, on the skin-facing surface 23 of the top sheet 2, the thick parts 21 are formed to protrude towards a skin-facing side relative to the thin parts 22, and on the non-skin-facing surface 24 of the top sheet 2, the thick parts 21 are formed to protrude towards a non-skin-facing side relative to the thin parts 22. On the top sheet 2, the thick parts 21 and the thin parts 22 extend in parallel in the front-rear direction y and are alternately arranged in the width direction x. The thick parts 21 and the thin parts 22 are arranged to form a stripe pattern extending in the front-rear direction y. Thereby, the top sheet 2 is less likely to bend unintentionally in the front-rear direction y on both the skin-facing surface and the non-skin-facing surface. In the top sheet 2, the thick parts 21 and the thin parts 22 are arranged a stripe pattern extending in the front-rear direction y in this manner. The thick parts 21 and the thin parts 22 extend from the portion overlapping with the absorbent body 4 and extend forward beyond a front edge 41 of the absorbent body 4 and extend rearward beyond a rear edge 42 of the absorbent body 4.

The non-skin-facing surface 24, on which adhesive is arranged, of the top sheet 2 is bonded to a member that contacts on the non-skin-facing side by the adhesive. The non-skin-facing surface 24 of the top sheet 2 includes a first adhesive region 25 and a second adhesive region 26. The first adhesive region 25 is arranged at least so to overlap with the absorbent body 4, and the second adhesive region 26 is positioned anterior and posterior to the first adhesive region 25, and is arranged at least forward of the front edge 41 of the absorbent body 4 and rearward of the rear edge 42 of the absorbent body 4. In the first adhesive region 25, the adhesive is arranged to extend in a spiral pattern or a meandering pattern in the front-rear direction y, and in the second adhesive region 26, the adhesive is entirely arranged over the second adhesive region.

As described above, in the absorbent article 1, the thick parts 21 and the thin parts 22 of the top sheet 2 extend forward and backward from the portion that overlaps with the absorbent body 4. The non-skin-facing surface 24 of the top sheet 2 includes the second adhesive region 26 over that adhesive is entirely arranged, and the second adhesive region 26 is positioned at least forward and rearward of the absorbent body 4. With this configuration, the shape retention of the absorbent article 1 in the planar direction is enhanced at its front and rear portions. With this configuration, the absorbent article 1 tends to be maintained in a flat state when a user handles the absorbent article 1 by grasping the front and rear end portions. As a result, the handleability of the absorbent article 1 becomes improved when being attached to underwear or placed on a wearer's crotch. Furthermore, since the second adhesive regions 26 at the front and rear of the absorbent body 4 of the top sheet 2 are entirely coated with the adhesive, the surface pattern of the thick parts 21 and the thin parts 22 appears to be more pronounced in the second adhesive regions 26 of the top sheet 2. Therefore, when handling the absorbent article 1, it is easier to visually distinguish between the portion where the absorbent body 4 is present and the regions where the absorbent body 4 is not present, making it easier to accurately position the absorbent article 1 on the underwear or on the crotch.

As described above, since the top sheet 2 includes thick parts 21 and the thin parts 22 that are arranged in a stripe pattern extending in the front-rear direction y, diffusibility of urine and the like in the front-rear direction y is increased on the skin-facing surface 23 of the top sheet 2, however, diffusibility of that in the width direction x tends to be suppressed. The stripe pattern of ridges and grooves that extend in the front-rear direction x and are formed by the thick parts 21 and the thin parts 22 appears to be more pronounced particularly in the second adhesive region 26. In contrast, since in the first adhesive region 25, adhesive is arranged in a spiral pattern or a meandering pattern that extends in the front-rear direction y, the top sheet 2 is intermittently adhered in the front-rear direction y within the first adhesive region 25. As a result, the top sheet 2 tends to be more likely to develop wrinkles extending in the width direction x in the first adhesive region 25. Furthermore, on the skin-facing surface 23 of the top sheet 2, the thin parts 22 are recessed to be grooves extending in the front-rear direction y. Also, in the first adhesive region 25, shallow and deep irregularities at the grooves tend to be formed alternately in the front-rear direction y. As a result, in the first adhesive region 25, both the diffusion of urine and the like in the front-rear direction y and the diffusion in the width direction x are ensured on the skin-facing surface 23 of the top sheet 2. In particular, since the first adhesive region 25 is arranged to overlap with the absorbent body 4, urine and the like excreted on the skin-facing surface 23 of the top sheet 2 are more likely to diffuse in both the front-rear direction y and the width direction x in the portion overlapping with the absorbent body 4, allowing the urine and the like to rapidly pass through the top sheet 2 and be suitably absorbed by the absorbent body 4. In addition, in the first adhesive region 25 of the top sheet 2, the stripe pattern of the thick parts 21 and the thin parts 22 that extend in the front-rear direction y becomes less noticeable at the irregularities, giving the top sheet 2 a softer appearance and texture.

As shown in FIG. 4, in the first adhesive region 25, an adhesive part applied in a spiral pattern or a meandering pattern needs only to be formed to extend in the front-rear direction y as a whole. That is, the direction of extension of the repeating pattern of spiral curves or meandering curves need only substantially coincide with the front-rear direction y. The adhesive part means a part where adhesive is actually arranged, and in the first adhesive region 25, the adhesive part is formed to extend spirally or meanderingly in the front-rear direction y. The first adhesive region 25 is defined as a region that surrounds the adhesive part applied in a spiral pattern or a meandering pattern.

The adhesive part of a spiral pattern can be formed, for example, by applying adhesive using a spiral coating method, while the adhesive part of a meandering pattern can be formed, for example, by applying adhesive using an omega coating method. The meandering pattern includes, for example, a sinusoidal pattern, a repeating omega-shaped pattern, and the like. The width range of the adhesive part of the spiral or meandering pattern is preferably 5 mm or more, more preferably 10 mm or more, and is preferably 25 mm or less, more preferably 20 mm or less.

Two or more rows of adhesive parts extending meanderingly or spirally in the front-rear direction y are preferably arranged in the width direction x in the first adhesive region 25. In this case, the adhesive parts arranged in the two or more rows are arranged in the width direction x, and adjacent adhesive parts may partially overlap with each other, and adjacent adhesive parts may be separated from each other in the width direction x. The latter arrangement of the adhesive parts is preferable because urine or the like tends to easily pass through the top sheet 2 and be rapidly absorbed by the absorbent body 4. In the first adhesive region 25, the adhesive parts arranged in the two or more rows in the width direction x are preferably spaced apart from each other by 10 mm or less, more preferably 8 mm or less, even more preferably 5 mm or less, and preferably 1 mm or more, and more preferably 2 mm or more.

In the second adhesive region 26 is a full-surface adhesive coating region; that is, the adhesive coating is entirely arranged over the second adhesive region 26. The adhesive application method for the second adhesive region 26 is not particularly limited, as long as the full-surface adhesive coating is arranged over the entire area of the region. For example, adhesive may be applied in a planar coating, or may be applied in a continuous linear coating without gaps to achieve an entire area coating. Alternatively, linear adhesive may be applied over a planar adhesive coating. In either case, the region over that the adhesive is entirely arranged becomes the second adhesive region 26.

In the first adhesive region 25 and the second adhesive region 26, the top sheet 2 is adhered or bonded to a member that contacts on the non-skin-facing surface of the top sheet. In the portion where the top sheet 2 overlaps with the absorbent body 4, the top sheet 2 may be adhered to the absorbent body 4. If a second sheet is placed between the top sheet 2 and the absorbent body 4, the top sheet 2 may be adhered to the second sheet. In the region where the top sheet 2 does not overlap with the absorbent body 4, the top sheet 2 may be bonded to the back sheet 3. If a base sheet 5 is placed between the back sheet 3 and the absorbent body 4, the top sheet 2 may be bonded to the base sheet 5. In the portion where the top sheet 2 overlaps with the absorbent body 4, it is preferable that the top sheet 2 is directly adhered to the absorbent body 4 and no other member is disposed between the top sheet 2 and the absorbent body 4. As a result, wrinkles extending in the width direction x tend to form in the first adhesive region 25 of the top sheet 2. Note that, from the viewpoint of preventing leakage in the front-rear direction, it is preferable that the top sheet 2 and the back sheet 3 are directly bonded to each other at least at front and rear edges of the absorbent article 1, as described below.

The thick parts 21 and the thin parts 22 of the top sheet 2 are described in detail. The top sheet 2 is preferably a nonwoven fabric. The thick parts 21 and the thin parts 22 are preferably formed by embossing the nonwoven fabric to thin the nonwoven fabric at the embossed portions. That is, in the top sheet 2, the thin parts 22 are preferably the embossed portions. This ensures shape retention in the thin parts 22 of the top sheet 2, which makes the top sheet 2 less likely to bend unintentionally in the front-rear direction y and also less likely to bend unintentionally in the width direction x at the thin parts 22. Accordingly, shape retention of the top sheet 2 is enhanced in the planar direction. The embossed portion of each of the thin parts 22 may be a heat-embossed portion, i.e., a portion embossed with heating, or a non-heat-embossed portion, i.e., a portion embossed without heating, and the former heat-embossed portion is more preferred. That is, it is more preferred that the nonwoven fabric is heat-embossed at the thin parts 22 of the top sheet 2. With this configuration, it easier to ensure shape retention at the thin parts 22 of the top sheet 2.

To form the thin parts 22 recessed relative to the thick parts 21 on both the skin-facing surface 23 and the non-skin-facing surface 24 of the top sheet 2, as one example, an embossing roll having ridges and grooves that circumferentially extend in parallel and that are arranged alternately in the axial direction on its circumferential surface and a flat roll may be prepared, and a nonwoven fabric may be sandwiched between the embossing roll and the flat roll to be embossed. As another example, two embossing rolls may be prepared, each having ridges and grooves that circumferentially extend in parallel and that are arranged alternately in the axial direction on each circumferential surface. The two embossing rolls may be arranged with the ridges of the two embossing rolls facing each other, and a nonwoven fabric may be sandwiched between the two embossing rolls to be embossed. In the case where the thin parts 22 are heat-embossed portions, one or both of the two rolls may be heated rolls. In the above example, it is preferable to use the two rolls that are heated.

The thickness T1 of each of the thick parts 21 of the top sheet 2 is preferably 0.3 mm or more, more preferably 0.4 mm or more. This configuration ensures the thickness of the top sheet 2, and enhances shape retention of the top sheet 2 in the front-rear direction y. Meanwhile, the thickness T1 of the thick part 21 of the top sheet 2 is preferably 1.8 mm or less, more preferably 1.5 mm or less. This configuration prevents the top sheet 2 from becoming excessively thick, and the liquid permeability of the top sheet 2 is easily ensured.

The arrangement pitch of the thick parts 21 that are arranged side by side in the width direction x is preferably 1 mm or more, more preferably 2 mm or more, and preferably less than 5 mm. The arrangement pitch of the thick parts 21 means a distance in the width direction x between the thickest part of a first thick part 21 and the thickest part of a second thick part 21 that is adjacent to the first thick part 21. The configuration of the thick parts 21 and the thin parts 22 results in enhancing the shape retention of the top sheet 2 in the front-rear direction y. The arrangement pitch of the thin parts 22 that are arranged side by side in the width direction x may be the same as the arrangement pitch of the thick parts 21 described above.

In the top sheet 2, the thickness T1 of each of the thick parts 21 is preferably 3 times or more and 15 times or less the thickness T2 of each of the thin parts 22. When the thickness T1 of the thick part 21 is 3 times or more the thickness T2 of the thin part 22, the difference between the thickness T1 of the thick part 21 and the thickness T2 of the thin part 22 becomes large, and the top sheet 2 becomes less likely to bend unintentionally in the front-rear direction y and enhancing the shape retention of the top sheet 2 in the front-rear direction y. When the thickness T1 of the thick part 21 is 15 times or less the thickness T2 of the thin part 22, the thickness at the thin part 22 is ensured, and the top sheet 2 becomes less likely to bend unintentionally in the width direction x and enhancing the shape retention of the top sheet 2 in the width direction x. The thickness T1 of the thick part 21 is preferably 12 times or less, more preferably 10 times or less the thickness T2 of the thin part 22.

The thickness T1 of the thick part 21 and the thickness T2 of the thin part 22 of the top sheet 2 can be determined by peeling the top sheet 2 from the absorbent article 1, cutting the top sheet along the width direction x, and measuring the thickness T1 of the thick part 21 and the thickness T2 of the thin part 22 at the cut surface. The thickness T1 of the thick part 21 is determined by measuring the thickness at the thickest part of the thick part 21 located between adjacent thin parts 22 in the width direction x. The thickness T2 of the thin part 22 is determined by measuring the thickness at the thinnest part of the thin part 22 located between adjacent thick parts 21 in the width direction x. Cutting of the top sheet 2 along the width direction x is performed at three positions: a first position that is within 1 cm forward of the front edge 41 of the absorbent body 4, a second position that is within 1 cm rearward of the rear edge 42 of the absorbent body 4, and a third position that is a center position in the front-rear direction y and at the center position the top sheet 2 overlaps with the absorbent body 4; the thicknesses of five or six thick parts 21 and the thin parts 22 that are positioned in a central portion in the width direction x are measured at each of these three positions; and their average values are calculated to obtain the thickness T1 of the thick part 21 and the thickness T2 of the thin part 22. The thicknesses of the thick part 21 and the thin part 22 can be conveniently measured from an image of the cut surface of the top sheet 2.

In the top sheet 2, the thick parts 21 and the thin parts 22 are preferably arranged over a larger area that is forward of the front edge 41 of the absorbent body 4 and preferably arranged over a larger area that is rearward of the rear edge 42 of the absorbent body 4. In the area that is forward of the front edge 41 of the absorbent body 4 in the top sheet 2, the lengths (maximum lengths) in the front-rear direction y of the thick parts 21 and the thin parts 22 arranged in a stripe pattern are preferably 50% or longer, more preferably 70% or longer, and even preferably 80% or longer of the length in the front-rear direction y of that area. It is particularly preferable that the thick parts 21 and the thin parts 22 arranged in a stripe pattern extend in parallel entirely in the front-rear direction y in the area forward of the front edge 41 of the absorbent body 4 of the top sheet 2. In the area rearward of the rear edge 42 of the absorbent body 4 of the top sheet 2, the lengths in the front-rear direction y of the thick parts 21 and the thin parts 22 arranged in a stripe pattern are preferably 50% or longer, more preferably 70% or longer, and even preferably 80% or longer of the length in the front-rear direction y of that area. It is particularly preferable that the thick parts 21 and the thin parts 22 that are arranged in a stripe pattern extend in parallel entirely in the front-rear direction y in the area rearward of the rear edge 42 of the absorbent body 4 of the top sheet 2. It is particularly preferable that the thick parts 21 and the thin parts 22, that are arranged in a stripe pattern, extend in parallel in the front-rear direction y and are arranged alternately in the width direction x in the area rearward of the rear edge 42 of the absorbent body 4 of the top sheet 2.

In the top sheet 2, it is preferable that the thick parts 21 and the thin parts 22 arranged in a stripe pattern are provided over a larger area that overlaps with the absorbent body 4. In the portion, that overlaps with the absorbent body 4, of the top sheet 2, the lengths (maximum lengths) in the front-rear direction y of the thick parts 21 and the thin parts 22 arranged in a stripe pattern are preferably 20% or longer, more preferably 40% or longer, even more preferably 60% or longer, and still even more preferably 80% or longer of the length in the front-rear direction y of the absorbent body 4. It is particularly preferable that each of the thick parts 21 and the thin parts 22 extends continuously in the front-rear direction y from the front edge 41 to the rear edge 42 of the absorbent body 4.

It is preferable that the thick parts 21 and the thin parts 22 extend continuously in the front-rear direction y from a front edge to a rear edge of the top sheet 2. With this configuration, the shape retention in the planar direction of the absorbent article 1 tends to be enhanced over the entire length in the front-rear direction y, and the absorbent article 1 achieves better handleability when being grasped at front and rear end portions of the absorbent article 1.

The entire width in the width direction x of the stripe pattern in that the thick parts 21 and the thin parts 22 are alternately arranged is preferably 80% or wider, more preferably 90% or wider of the width in the width direction x of the absorbent body 4, and may even be wider than the width in the width direction x of the absorbent body 4. In addition, the entire width in the width direction x of the stripe pattern in that the thick parts 21 and the thin parts 22 are alternately arranged is preferably 80% or wider, more preferably 90% or wider of the width in the width direction x of the top sheet 2. With this configuration of the absorbent article 1, shape retention tends to be ensured in the planar direction over a larger range in the width direction x of the absorbent article 1.

The mass per unit area of the top sheet 2 is preferably 20 g/m² or more and 35 g/m² or less. With this configuration of the top sheet 2, the shape retention of the top sheet 2 in the planar direction is easily ensured, and also, liquid permeability of the top sheet 2 tends to be ensured. The mass per unit area of the top sheet 2 is preferably 22 g/m2 or more, more preferably 25 g/m2 or more, and preferably 32 g/m² or less, more preferably 30 g/m² or less.

The first adhesive region 25 and the second adhesive region 26 of the top sheet 2 are described in detail. On the non-skin-facing surface 24 of the top sheet 2, the first adhesive region 25 is arranged to overlap at least with the absorbent body 4, and may also be arranged in a region that does not overlap with the absorbent body 4. It is preferable that the first adhesive region 25 is arranged over a larger area of the portion overlapping with the absorbent body 4. In the portion overlapping with the absorbent body 4, the length of the first adhesive region 25 in the front-rear direction y is preferably 60% or longer, more preferably 70% or longer, even more preferably 80% or longer of the length of the absorbent body 4 in the front-rear direction y.

On the non-skin-facing surface 24 of the top sheet 2, the second adhesive region 26 is arranged at least in a region that does not overlap with the absorbent body 4, and may also be arranged in a portion that overlaps with the absorbent body 4. On the non-skin-facing surface 24 of the top sheet 2, the second adhesive region 26 is preferably arranged over a larger area forward of the front edge 41 of the absorbent body 4, and is preferably arranged over a larger area rearward of the rear edge 42 of the absorbent body 4.

In one embodiment of the present invention, on the non-skin-facing surface 24 of the top sheet 2, it is preferable that the second adhesive region 26 includes at least a first region arranged at least forward of the front edge 41 of the absorbent body 4 to a front edge of the top sheet 2 and a second region arranged at least rearward of the rear edge 42 of the absorbent body 4 to a rear edge of the top sheet 2. When the second adhesive region 26 is further arranged from a portion overlapping with at least one of a front end part and a rear end part of the absorbent body 4, the front end part and/or the rear end part of the absorbent body 4 is/are bonded in the planar direction between the top sheet 2 and the back sheet 3, resulting in a structure that is more resistant to leakage forward of the front edge 41 of the absorbent body 4 and/or rearward of the rear edge 42 of the absorbent body 4. The first adhesive region 25 is disposed between the first region and the second region of the second adhesive region 26 in the front-rear direction y of the absorbent article 1. The first adhesive region 25 is preferably arranged to overlap with 80% or more of the absorbent body 4 when viewed in a plan.

In the region forward of the front edge 41 of the absorbent body 4 on the top sheet 2, the length of the second adhesive region 26 in the front-rear direction y is preferably 50% or longer, more preferably 70% or longer, even more preferably 80% or longer, and particularly preferably 100% of the length of the region forward of the front edge 41 of the absorbent body 4 in the front-rear direction y. That is, it is particularly preferable that the second adhesive region 26 extends up to the front edge of the top sheet 2. As the front edge of the top sheet 2 is directly and securely bonded to the front edge of the back sheet 3, it is expected to prevent leakage forward.

In the region rearward of the rear edge 42 of the absorbent body 4 on the top sheet 2, the length of the second adhesive region 26 in the front-rear direction y is preferably 50% or longer, more preferably 70% or longer, even more preferably 80% or longer, and particularly preferably 100% of the length of the region rearward of the rear edge 42 of the absorbent body 4 in the front-rear direction y. That is, it is particularly preferable that the second adhesive region 26 extends up to the rear edge of the top sheet 2. As the rear edge of the top sheet 2 is directly and securely bonded to the rear edge of the back sheet 3, it is expected to prevent leakage rearward.

The second adhesive region 26 is preferably arranged from the portion that overlaps with the absorbent body 4 to the region that does not overlap with the absorbent body 4 in a plan view. Specifically, the second adhesive region 26 is preferably arranged from the portion that overlaps with at least one of the front end part and the rear end part of the absorbent body 4 to the region that does not overlap with the absorbent body 4. This configuration enhances the shape retention of the absorbent article 1 in the planar direction from the portion that overlaps with the at least one of the front end part and the rear end part of the absorbent body 4 to the region that does not overlap with the absorbent body 4. As a result, when a user handles the absorbent article 1 by grasping the front end part or the rear end part of the absorbent article 1, the absorbent article 1 is less likely to bend at the front edge 41 or the rear edge 42 of the absorbent body 4. Accordingly, the handleability of the absorbent article 1 is improved. In the absorbent article 1, the second adhesive region 26, that extends from the portion that overlaps with the absorbent body 4 to the region that does not overlap with the absorbent body 4, is more preferably arranged at both the front end part and the rear end part of the absorbent body 4.

In the case that the second adhesive region 26 is arranged as described above, the length in the front-rear direction y of an overlapping portion, where the second adhesive region 26 overlaps with at least one of the front end part and the rear end part of the absorbent body 4 is preferably 20% or shorter of the length in the front-rear direction y of the absorbent body 4. Since diffusion of urine and the like in the width direction x in the front-rear direction y tends to be decreased, it is preferable that the length in the front-rear direction y of the overlapping portion of the second adhesive region 26 is 20% or shorter of the length of the absorbent body 4. With this configuration, the diffusion of urine and the like in the width direction x on the skin-facing surface 23 of the top sheet 2 is sufficiently ensured in the area where the top sheet 2 overlaps with the absorbent body 4, allowing urine and the like to rapidly pass through the top sheet 2 and be suitably absorbed in the absorbent body 4. The second adhesive region 26 may have an overlapping portion that overlaps with the front end part of the absorbent body, and the length in the front-rear direction y of the overlapping portion may be 20% or shorter of the length in the front-rear direction y of the absorbent body 4. Also, the second adhesive region 26 may have an overlapping portion that overlaps with the rear end part of the absorbent body, and the length in the front-rear direction y of the overlapping portion may be 20% or shorter of the length in the front-rear direction y of the absorbent body 4. Furthermore, the second adhesive region 26 may have overlapping portions that overlap with the front end part and the rear end part of the absorbent body 4, and the total length in the front-rear direction y of the overlapping portions may be 20% or shorter of the length in the front-rear direction y of the absorbent body 4. Preferably, in the second adhesive region 26 that overlaps with the absorbent body 4, the length in the front-rear direction y of the overlapping portion of the second adhesive region 26 is 20% or shorter of the length in the front-rear direction y of the absorbent body 4. That is, the total length in the front-rear direction y of the overlapping portion, that overlaps with the absorbent body 4, of the second adhesive region 26 is preferably 20% or shorter of the length, in the front-rear direction y, of the absorbent body 4. Furthermore, it is more preferable that the length in the front-rear direction y of the overlapping portion, that overlaps with a front half of the absorbent body 4, of the second adhesive region 26 is 10% or shorter of the length in the front-rear direction y of the absorbent body 4, and the length in the front-rear direction y of the overlapping portion, that overlaps with a rear half of the absorbent body 4, of the second adhesive region 26 is 10% or shorter of the length in the front-rear direction y of the absorbent body 4.

The second adhesive region 26 may be located spaced apart from the first adhesive region 25 in the front-rear direction y, or may be located in contact with the first adhesive region 25 in the front-rear direction y, as long as the second adhesive region 26 is positioned anterior and posterior to the first adhesive region 25. However, the second adhesive region 26 is preferably arranged in contact with the first adhesive region 25 in the front-rear direction y, and preferably without a non-adhesive region between the first adhesive region 25 and the second adhesive region 26 on the non-skin-facing surface 24 of the top sheet 2. If there is a non-adhesive region between the first adhesive region 25 and the second adhesive region 26, a large wrinkle extending in the width direction x tends to occur in such a non-adhesive region of the top sheet 2 and hinder the diffusion of urine and the like in the front-rear direction y. Meanwhile, when the second adhesive region 26 is arranged to be positioned in contact with the first adhesive region 25 in the front-rear direction y, the diffusion of urine and the like in the front-rear direction y on the skin-facing surface 23 of the top sheet 2 can be more effectively ensured.

The width of the first adhesive region 25 in the width direction x and the width of the second adhesive region 26 in the width direction x are not particularly limited; however, it is preferable that the width of the second adhesive region 26 in the width direction x is wider than the width of the first adhesive region 25 in the width direction x. With this configuration, it is easier for a user to grasp and handle the front and rear end portions of the absorbent article 1. In the absorbent article 1, shape retention in the planar direction is enhanced in the second adhesive region 26 that is positioned forward and rearward of the absorbent body 4, and thus, it is easier for a user to grasp the front and rear end portions of the absorbent article 1 and the absorbent article 1 tends to be maintained in a flat state while it is grasped at the front end portion and the rear end portion. Therefore, the second adhesive regions 26 with the wider width in the width direction x, the handleability of the absorbent article 1 can be improved when a user grasps its front end portion and the rear end portion.

The width of the first adhesive region 25 in the width direction x is preferably 50% or wider, more preferably 60% or wider of the width of the top sheet 2 in the width direction x. Furthermore, the width of the first adhesive region 25 in the width direction x is preferably 30% or wider, more preferably 35% or wider, even more preferably 70% or wider of the width of the absorbent body 4 in the width direction x. In order to maintain the shape of the absorbent article 1, the first adhesive region 25 may be disposed to be up to 110% of width of the absorbent body 4 in the width direction x.

The width of the second adhesive region 26 in the width direction x is preferably 70% or wider, more preferably 80% or wider of the width of the top sheet 2 in the width direction x. Furthermore, the width of the second adhesive region 26 in the width direction x is preferably 40% or wider, more preferably 45% or wider of the width of the absorbent body 4 in the width direction x.

From the viewpoint that the effect of forming the thick parts 21 and the thin parts 22 of the top sheet 2 and of arranging the second adhesive region 26, over that adhesive is entirely applied, to be positioned forward and rearward of the absorbent body 4 as described above can be more effectively exhibited, the absorbent article 1 preferably has the absorbent body 4 with at least one of the front edge 41 and the rear edge 42 being straight, and more preferably both the front edge 41 and rear edge 42 of the absorbent body 4 are straight. When the front edge 41 and rear edge 42 of the absorbent body 4 are straight, the absorbent article 1 tends to be easily bent at the edges; however, the top sheet 2 is formed and arranged as described above, the effect of the absorbent article 1 becoming less likely to bend at the front edge 41 and the rear edge 42 of the absorbent body 4 is more readily achieved. In this case, the front edge 41 and the rear edge 42 of the absorbent body 4 are preferably formed to be straight to extend in the width direction x. An example of such an absorbent body 4 is a rectangular-shaped absorbent body 4.

The length in the front-rear direction y of the portion, that is positioned forward of the front edge 41 of the absorbent body 4, of the top sheet 2 is preferably 2 cm or longer and 7 cm or shorter. The length in the front-rear direction y of the portion, that is positioned rearward of the rear edge 42 of the absorbent body 4, of the top sheet 2 is preferably 2 cm or longer and 7 cm or shorter. With this configuration of the absorbent article 1, the effect of arrangement of the top sheet 2 on that the thick parts 21 and the thin parts 22 extend in parallel in the front-rear direction y in a stripe pattern and the second adhesive region 26, over that adhesive is entirely arranged, is located forward and rearward of the absorbent body 4 is more reliably achieved. That is, shape retention of the absorbent article 1 tends to be enhanced in the planar direction in the front end portion and rear end portion of the absorbent article 1. Accordingly, it is easier for a user to grasp the front and rear end portions of the absorbent article 1 and the absorbent article 1 tends to be maintained in a flat state while it is grasped at the front and rear end portions. The length in the front-rear direction y of the portion, that is positioned forward of the front edge 41 of the absorbent body 4, is more preferably 2.5 cm or longer, and more preferably 6 cm or shorter, and the length in the front-rear direction y of the portion that is positioned rearward of the rear edge 42 of the absorbent body 4 is more preferably 2.5 cm or longer, and more preferably 6 cm or shorter.

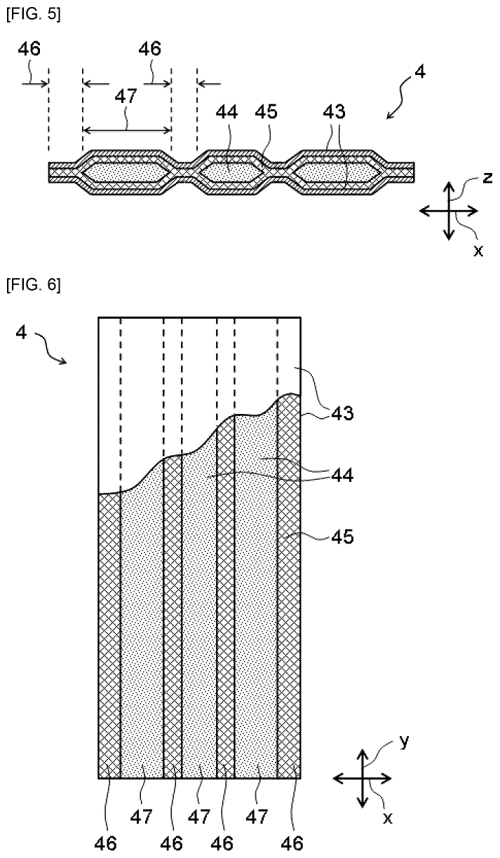

The top sheet 2 as described above is particularly preferably applied when the absorbent body 4 is a sheet-shaped absorbent body, especially when the absorbent body 4 is configured, as shown in FIGS. 5 and 6. The absorbent body 4 includes two or more sheet members 43, absorbent polymer 44 without a pulp fiber is disposed between the sheet members 43, and a sealed part 46 at which the sheet members 43 are bonded to each other and a non-sealed part 47 at which the sheet members 43 are not bonded to each other. The sealed parts 43 and the non-sealed parts 47 that extend in parallel in the front-rear direction y are arranged in a stripe pattern and the absorbent polymer 44 is disposed in the non-sealed part 47. FIG. 5 shows a cross-sectional view in the width direction x of the absorbent body 4 of a sheet-shaped absorbent body, and FIG. 6 shows a partially cutaway plan view of the absorbent body 4 of the sheet-shaped absorbent body as seen from the top sheet 2 side.

In the absorbent body 4 configured as described above, while the diffusibility of urine and the like in the front-rear direction y tends to be enhanced, the diffusibility in the width direction x tends to decrease. However, the top sheet 2 includes the first adhesive region 25 that is arranged in a region that overlaps with the absorbent body 4, and thus, in the first adhesive region 25, both the diffusion of urine and the like in the front-rear direction y and the diffusion in the width direction x are ensured on the skin-facing surface 23 of the top sheet 2. As a result, decrease in the diffusibility in the width direction x of the absorbent body 4 is expected to be compensated. This enhances the absorption performance of urine and the like by the absorbent body 4.

Furthermore, although the absorbent body 4 configured as described above may be susceptible to leakage of the absorbent polymer 44 from its front and rear ends, this risk can be eliminated by the second adhesive region 26 on the non-skin-facing surface 24 of the top sheet 2. That is, since the non-skin-facing surface 24 of the top sheet 2 has the second adhesive region 26, over which adhesive is entirely arranged to form an entirely adhesive-coated region, and the second adhesive region 26 is arranged at least forward of the front edge 41 of the absorbent body 4 and at least rearward of the rear edge 42 of the absorbent body 4, it is possible to prevent particles of the absorbent polymer 44 from spilling out of the absorbent body 4 through the front and rear ends of the absorbent body 4. The second adhesive region 26 is preferably arranged to overlap with the front edge 41 and/or the rear edge 42 of the absorbent body 4 by two to 20 mm inward in the front-rear direction y. For example, in the case where two or more sheet-shaped absorbent bodies are provided as the absorbent body 4, when the second adhesive region 26 overlaps with the front edge 41 and/or the rear edge 42 of the absorbent body 4 by 5 mm or more inward in the front-rear direction y, movement of the absorbent polymer in the front and/or the rear direction is suppressed by the second adhesive region 26 in expanding the absorbent polymer during use of the absorbent article 1, making it easier to prevent leakage forward and/or backward.

The configuration of the absorbent article shown in FIGS. 5 and 6 is described in detail. The absorbent body 4 includes two or more sheet members 43, and an absorbent polymer 44 without a pulp fiber is disposed between the sheet members 43. That is, in the absorbent body 4, one of the sheet members 43 is disposed at a side closer to the skin-facing surface of the absorbent body 4 and the other of the sheet members 43 is disposed at a side closer to the non-skin-facing surface of the absorbent body 4, and the absorbent polymer 44 is disposed between the sheet members 43, but a pulp fiber is not disposed between the sheet members 43. It is noted that the presence of a pulp fiber inevitably mixed in between the sheet members 43 during manufacturing is allowed. With the configuration of the absorbent body 4 above, the absorbent body 4 can be thinned while having sufficient absorption capacity.

The shape (planar shape) of the absorbent body 4 is not particularly limited. Examples of the shape of the absorbent body 4 include a substantially rectangular shape, an hourglass shape, a gourd shape, a battledore shape and others.

As the absorbent polymer 44, a polyacrylic absorbent polymer such as sodium polyacrylate; a starch-based absorbent polymer such as starch-acrylonitrile graft copolymer, starch-acrylic acid graft copolymer and starch-acrylamide graft copolymer; a polyvinyl alcohol absorbent polymer such as crosslinked polyvinyl alcohol; or the like can be used. As the absorbent polymer 44, a polyacrylic absorbent polymer such as sodium polyacrylate is preferably used, in view of high liquid absorption ability.

The sheet member 43 is liquid permeable, and a nonwoven fabric made from hydrophilic fibers such as cellulose, rayon and cotton; a nonwoven fabric which is formed from hydrophobic fibers such as polyolefin (e.g., polypropylene and polyethylene), polyester (e.g., PET) and polyamide (e.g., nylon), and in which the hydrophobic fibers are hydrophilized with a surfactant on the surface thereof; or the like can be used, for example. As the sheet member 43, a woven fabric, a knitted fabric, a plastic film having holes or the like may be also used. Preferably, a nonwoven fabric sheet member, namely, a nonwoven fabric sheet is used, as the sheet member 43.

For the sheet member 43, a first sheet member may be disposed at a side closer to the skin-facing surface of the absorbent article and a second sheet member may be disposed at a side closer to the non-skin-facing surface of the absorbent article, or a single sheet member may be folded back, and one side of the folded single sheet member may serve as the first sheet member at a side closer to the skin-facing surface and the other side of the folded single sheet member may serve as the second sheet member at a side closer to the non-skin-facing surface of the absorbent article.

The absorbent body 4 includes a sealed part 46 in which the sheet members 43 are bonded to each other and a non-sealed part 47 in which the sheet members 43 are not bonded to each other, and the absorbent polymer 44 is disposed in the non-sealed part 47. The two or more sealed parts 46 and two or more non-sealed parts 47 extend in parallel in the front-rear direction y and are alternately arranged in the width direction x. The sealed part 46 is formed by bonding the sheet members 43 to each other with adhesive or by welding (heat sealing, ultrasonic welding, or the like). The non-sealed part 47 is defined as a part where the sheet members 43 are not bonded to each other, and a part other than the sealed part 46 corresponds to the non-sealed part 47. As an absorbent body 4, an absorbent polymer particle may also be present in the sealed part 46.

In FIG. 5, adhesive is applied to the sheet member 43 to form an adhesive layer 45, and the absorbent polymer 44 is bonded to the sheet member 43 by the adhesive layer 45 in the non-sealed part 47. The adhesive layer 45 may be provided on an inner surface of at least one of the sheet member 43 that is positioned at a side closer to the skin-facing surface and the sheet member 43 that is positioned at a side closer to the non-skin-facing surface. The adhesive layer 45 is preferably provided on inner surfaces of both sheet members 43 positioned at the side closer to the skin-facing surface and positioned at the side closer to the non-skin-facing surface. At least a part of the absorbent polymer 44 may be bonded to the adhesive layer 45. When the absorbent polymer 44 is bonded to the sheet member 43 by the adhesive layer 45, particles of the absorbent polymer 44 hardly move between the sheet members 43 prior to absorption of the absorbent polymer 44, and the ability of the absorbent body 4 to absorb urine and the like is easily secured. Furthermore, even after the absorbent polymer 44 has absorbed liquid, gelled absorbent polymer 44 would be less likely to move between the sheet members 43, and as a result, it hardly occurs to give a wearer uncomfortable feeling due to the lump-formed absorbent polymer 44.

It is preferable that the adhesive layer 45 does not inhibit water absorption or swelling of the absorbent polymer 44 while bonding the absorbent polymer 44 to the sheet members. In order to form such an adhesive layer 45, the adhesive layer 45 is preferably formed by a curtain spray method, a spiral coating method, an omega coating method, a coater method or the like.

The sealed part 46 preferably maintains the bonding between the sheet members 43 even after absorption of the absorbent polymer 44. With this configuration, the ability of the absorbent body 4 to absorb urine and the like tends to be secured even after initial liquid absorption of the absorbent polymer 44. From this standpoint of view, the sealed part 46 is preferably formed by bonding the sheet members 43 to each other with an adhesive such as a rubber-based adhesive or a styrene-based elastomer or formed by welding the sheet members 43 to each other.

In the absorbent body 4, the sealed parts 46 and the non-sealed parts 47 that extend in parallel in the front-rear direction y are arranged in a stripe pattern. The sealed parts 46 are preferably arranged to extend over the entire length in the front-rear direction y. With this configuration of the absorbent body 4, urine and the like tends to be easily diffused in the front-rear direction y at the sealed parts 46, and also, urine and the like reaching the absorbent body 4 through the top sheet 2 tends to be further diffused and absorbed rapidly in the absorbent body 4. Two or more non-sealed parts 47 are preferably arranged side by side in the width direction x, and it is more preferable that three or more non-sealed parts are arranged. Three or more sealed parts 46 are preferably arranged side by side in the width direction x, and it is more preferable that four or more sealed parts are arranged.

The width of each of the sealed parts 46 in the width direction x is preferably 1 mm or wider, more preferably 2 mm or wider, and preferably 10 mm or narrower. The width in the width direction x of each of the sealed parts 46 that are located at the outermost positions in the width direction x is preferably wider than the width in the width direction x of each of the sealed parts 46 that are located inward of the sealed parts 46 that are located at the outermost positions in the width direction x. The width of each of the sealed parts 46 that are located at the outermost positions in the width direction x is preferably 2 mm or wider and 10 mm or narrower. The width in the width direction x of each of the sealed parts 46 that are located inward in the width direction x is preferably narrower than the width of the sealed part 46 that is located at the outermost position in the width direction x, and is preferably 1 mm or wider and 5 mm or narrower. With the above configuration, when the absorbent polymer 44 expands during use of the absorbent article 1, movement and expansion of the absorbent polymer 44 in the width direction x is moderately suppressed without hindering its swelling.

The width of each of the non-sealed parts 47 in the width direction x is preferably 5 mm or wider, more preferably 8 mm or wider, and preferably 50 mm or narrower, more preferably 30 mm or narrower, even more preferably 20 mm or narrower. The width in the width direction x of each of the non-sealed parts 47 located at the outermost positions in the width direction x is preferably wider than the width in the width direction x of each of the non-sealed part 47 located inward of the non-sealed parts 47 located at the outermost positions in the width direction X.

The amount of the absorbent polymer 44 disposed in the non-sealed parts 47 of an absorbent layer is not particularly limited, and is preferably 100 g/m² or more, more preferably 150 g/m² or more, and preferably 400 g/m² or less. When the absorbent polymer 44 is disposed in the non-sealed parts 47 of the absorbent layer in the amount of 100 g/m² or more, absorption capacity of the absorbent body 4 can be easily increased. Meanwhile, when the absorbent polymer 44 is disposed in the non-sealed parts 47 of the absorbent layer in the amount of 400 g/m² or less, the bonding between the sheet members 43 in the sealed parts 46 is easily maintained even when the absorbent polymer 44 absorbs liquid.

### REFERENCE SIGNS LIST

1: absorbent article
2: top sheet
21: thick part
22: thin part
23: skin-facing surface
24: non-skin-facing surface
25: first adhesive region
26: second adhesive region
3: back sheet
4: absorbent body
41: front edge
42: rear edge
43: sheet member
44: absorbent polymer
45: adhesive layer
46: sealed part
47: non-sealed part
5: base sheet
6: standing flap
7: side sheet
8: elastic member for standing flap

1. An absorbent article having a front-rear direction and a width direction, comprising a top sheet, a back sheet and an absorbent body disposed between the top sheet and the back sheet:
the top sheet comprising thick parts and thin parts that extend in parallel in the front-rear direction and are arranged alternately in the width direction;
the thin parts that are recessed relative to the thick parts on a skin-facing surface of the top sheet, and the thin parts are recessed relative to the thick parts on a non-skin-facing surface of the top sheet;
the thick parts and the thin parts extending from a portion that overlaps with the absorbent body and extending forward beyond a front edge of the absorbent body and extending rearward beyond a rear edge of the absorbent body; and
the non-skin-facing surface of the top sheet comprising a first adhesive region in that adhesive is arranged to extend in a spiral pattern or a meandering pattern in the front-rear direction, and a second adhesive region over that adhesive is entirely arranged, wherein the first adhesive region is arranged at least to overlap with the absorbent body, and the second adhesive region is positioned anterior and posterior to the first adhesive region, and is arranged at least forward of the front edge of the absorbent body and at least rearward of the rear edge of the absorbent body.

2. The absorbent article according to claim 1, wherein
the second adhesive region is arranged from a portion that overlaps with at least one of a front end part and a rear end part of the absorbent body.

3. The absorbent article of claim 2, in the second adhesive region that is arranged from the portion overlapping with the at least one of the front end part and the rear end part of the absorbent body, wherein
the length in the front-rear direction of the overlapping portion is 20% or shorter of the length in the front-rear direction of the absorbent body.

4. The absorbent article according to claim 1, wherein
the second adhesive region comprises a first region extending at least from the front edge of the absorbent body to a front edge of the top sheet and a second region extending at least from the rear edge of the absorbent body to a rear edge of the top sheet.

5. The absorbent article according to claim 1, wherein
the front edge of the absorbent body is straight and the rear edge of the absorbent body is straight.

6. The absorbent article according to claim 1, wherein
the absorbent body comprises two or more I sheet members, and an absorbent polymer without a pulp fiber is disposed between the sheet members,
the absorbent body comprises a sealed part in that the sheet members are bonded to each other and a non-sealed part in that the sheet members are not bonded to each other, and the absorbent polymer is disposed in the non-sealed part, and
the sealed part and the non-sealed part extend in parallel in the front-rear direction and are arranged alternately in the width direction.

7. The absorbent article according to claim 1, wherein
the top sheet is a nonwoven fabric, and the nonwoven fabric is heat-embossed at the thin parts.

8. The absorbent article according to claim 1, wherein
the thick parts and the thin parts extend continuously in the front-rear direction from a front edge to a rear edge of the top sheet.
